(19) United States
(12) Patent Application Publication
(10) Pub. No.: US 2026/0201090 A1
(43) Pub. Date: Jul. 16, 2026

(54) FUNCTIONALIZED AND NON-FUNCTIONALIZED AMPHIPHILIC POLYMERS BASED ON POLYACRYLIC ACID FOR THE SOLUBILIZATION, ISOLATION AND MOLECULAR LABELLING OF MEMBRANE PROTEINS IN AQUEOUS MEDIA

(71) Applicant: CUBE BIOTECH GMBH, Monheim (DE)

(72) Inventors: Barbara Maertens, Köln (DE); Roland Fabis, Leverkusen (DE); Jan Kubicek, Köln (DE); Philipp Timo Hanisch, Langenfeld (DE); Sergej Balanda, Düsseldorf (DE); Michael Ekrelenz, Solingen (DE); Oliver Kortheuer, Düsseldorf (DE); Lukas Leon Lange, Düsseldorf (DE); Christoph Meisen, Langenfeld (DE)

(21) Appl. No.: 19/136,819

(22) PCT Filed: Dec. 4, 2023
(86) PCT No.: PCT/EP2023/084166

§ 371 Date: Jun. 6, 2025

Related U.S. Application Data
(60) Provisional application No. 63/430,135, filed on Dec. 5, 2022.

(51) Int. Cl. C08F 120/06; C08F 8/32; C08F 8/40; C12N 5/07

(52) U.S. Cl. CPC C08F 120/06; C08F 8/32; C08F 8/40; C12N 5/06; C08F 2438/03

(57) ABSTRACT

The present application relates to a water-soluble membrane protein solubilizing polymer having the general formula (1) wherein X is either the initiator fragment derived by the radical starter molecule or a functional group or a hydrogen atom; Y is a functional group, a hydrogen atom, a terminal RAFT agent usable for further functionalization or a conjugated molecule; a as well as b denotes for the relative number of repetitive units of the polymer and give rise to the molecular weight of the polymer; R₁ is a hydrophilic part of the polymer based on grafted proteinogenic and non-proteinogenic amino acids and amino acid derivatives, and R₂ is a hydrophobic part of the polymer. Furthermore, the method of preparing the polymer as well as its use are described.

### CROSS REFERENCE TO RELATED APPLICATIONS

This is a US national phase stage application under 35 U.S.C. § 371 of international patent application no. PCT/EP2023/084166 filed 4 Dec. 2023, which claims priority to U.S. provisional patent application No. 63/430,135.

### TECHNICAL FIELD

The invention relates to specifically defined polymers, a method for their preparation, their use for solubilizing and stabilizing membrane proteins as well as a kit for this use containing the polymers according to the present invention.

### BACKGROUND OF THE INVENTION

Membrane proteins are of great relevance for the biomedical research as about one third of all proteins are membrane proteins which are strongly involved in cellular communication, intermembrane transport of molecules and enzymatic reactions and thus can be targets for drugs. Due to their natural interaction with the alkyl chains of the cell membrane lipids and their structure with helices and sheets inserted into the hydrophobic c membrane, the isolation and stabilization of membrane proteins is challenging and amphiphilic detergents are needed for the disintegration of the cell membrane. Commonly, the surface of membrane proteins is characterized by the localization of hydrophobic amino acids in the regions inserted in the cell membrane and the localization of hydrophilic amino acids in the peripheral parts of membrane proteins. This Janus-like structure is the origin of the reduced solubility and thus stability of membrane proteins in aqueous media.

However, the functional activity of cell membrane proteins often relies on their natural lipid environment which is disrupted by solubilization using detergents. Furthermore, the structural stability of membrane proteins is disturbed after solubilization using common detergents (e.g., sodium dodecyl sulfate (SDS) or n-Dodecyl-beta-maltoside (DDM)), as they tend to aggregate especially during their expression and purification in large quantities.

The commercially available detergents (e.g. SDS) allow the dissolution of the membrane as well as the solubilization of the membrane protein of interest with high yield.[1] However, the solubilization using membrane proteins is often followed by denaturation and inactivation of the membrane protein and is limited due to the detergent concentration above the critical micelle concentration (CMC). Detergent concentrations in the regime of the CMC can have detrimental effects on the membrane protein function as they poorly mimic the lipids of the cell membrane.

Beside this, the presence of detergence in membrane protein solutions can have detrimental effects in further analyzation or application of the membrane proteins (e.g., cryo transmission electron spectroscopy or crystallization). Below the CMC the solubility of the membrane protein cannot be ensured due to the not fully detergent-covered hydrophobic patches of the protein transmembrane part.

Classic amphiphilic polymers called amphipols are used to overcome the challenges as they enable the stabilization of solubilized membrane proteins. The amphiphilic polymer directly binds with their hydrophobic parts to the likewise hydrophobic parts of the protein transmembrane region. This makes the use of detergent redundant since solubilization and stabilization of the membrane protein can be performed in a unique one-step process. The amphipol prototype A8-35 as described in WO1998027434A1 consists of a polyacrylic acid backbone grafted with octylamine (~25%), isopropylamine (~40%) and ~35% remaining carboxylic acid groups. However, the use of A8-35 requires in general the detergent-based solubilization of the membrane protein which can lead to a loss of function of the protein as the amphiphilic polymer surrounding the membrane protein does not mimic the unique natural environment of the cell membrane surrounding the membrane protein.

A new class of amphiphilic polymers published and patented are SMAs (Styrene Maleic-Acid, WO2006129127, WO2011004158), CyclApols (US20220119558A1) and AASTYs (Poly(acrylic acid-co-styrene).[2,3] The stabilization of these new polymers is based on their ability to mimic the natural environment of membrane proteins by the formation of small complexes called nanodiscs.[3]

The ring-like nanometer-sized nanodisc complexes consist of one or more amphiphilic polymer molecules which encircle a patch of cell membrane including the membrane protein.[4] Additionally, SMAs, CyclApols and AASTYs allow the solubilization and stabilization of membrane proteins without the use of detergents. However, the described polymers are limited with respect of either controlling the polymer length, the polydispersity, the monomer sequency, the flexibility of varying the hydrophobic or hydrophilic sidechains of the polymer and the ability of terminal functionalization of the polymer chain without affecting the polymerization efficiency itself.

The solubilization efficiency as well as the stability of the formed membrane protein containing nanodisc strongly depends on the characteristics of the used polymer. Polymers, like SMAs, CyclApols and AASTYs are stabilized by the electrostatic repulsion of the negatively charged polymer chain. The charges are generated by carboxylic groups which are implemented in the polymer by using monomers like acrylic acid or maleic acid and copolymerization with styrene in the case of SMA and AASTY. These nanodisc forming polymers bear the risk of aggregation as they are highly sensitive to the presence of divalent ions, low pH values (<6.5) and high ionic strength due to the covered negative charges of the stabilizing carboxylic acid group.[5] In contrast, polymers with highly charged polar groups like phosphates or sulfates [6] as well as flexible non-polar side chains, like described in this invention, enhance both the solubilization efficiency as well as the stability of the nanodiscs.

The length of the polymer as well as the polydispersity and the homogenous sequence of co-polymerized monomers like acrylic acid and styrene are critical factors for the solubilization itself or for the field of use like cryo transmission electron microscopy.[7] Thus, it is tremendously important to gain more control over the polymerization. Techniques used in the past, like the radical batch polymerization yield polymers with a high polydispersity (PDI >2) and an inhomogeneous distribution of the monomers in the polymer depending on their chemical properties.

Recently, the use of the reversible-addition-fragmentation chain-transfer polymerization called RAFT polymerization allows the synthesis of monodisperse polymers as shown with AASTYs (polydispersity <1.5).[8] The RAFT polymerization was first described in 1998 by Rizzardo et al. and is defined by being “a degenerate-transfer radical polymerization in which chain activation and chain deactivation involve a degenerative chain-transfer process which occurs by a two-step addition-fragmentation mechanism” (IUPAC definition).[9] A typical RAFT polymerization consists of one or more monomers (e.g., vinyl derivatives), a radical source (e.g., thermochemical initiators) and a RAFT agent (thiocarbonylthio compounds). Furthermore, RAFT synthesized polymers provide the opportunity of terminal functionalization due to the terminal thiocarbonylthio moiety which can be chemically substituted using different techniques like maleimide conjugation chemistry.[10] Terminal modifications of the polymers can beside other things include fluorophores or biomolecular tags like biotin for further analysis and purification of the synthesized nanodiscs containing membrane proteins.

### SUMMARY OF THE INVENTION

To overcome the drawbacks of current polymers for membrane solubilization, the object of the underlying and present invention is to provide a polymer, which is adaptive with respect of the used hydrophilic and hydrophobic side chains, is stable at high ionic strength, is resistant against divalent ions (e.g., Ca²⁺, >5 mM), is efficiently solubilizing membrane proteins, and is efficiently stabilizing membrane proteins. Additionally, the new invented polymer bears the important feature of selective solubility. Polymer not incorporated in nanodiscs can be removed by high-speed centrifugation which reduces the excess amount of polymer in the lysate enabling highly efficient purification using affinity resins and advanced downstream applications (e.g. cryo electron microscopy) which is negatively influenced by excess copolymers not incorporated in nanodiscs.

In another embodiment the polymer is monodisperse (PDI<1.25), more preferably with a pdi<1.2, even more preferably with a PDI<1.1, and is preferably functionalizable at the terminal end.

This has been achieved by the subject-matter of the independent claims. Preferred embodiments are defined in the dependent claims.

According to the present invention, there is provided amphiphilic vinyl derived polymers with the having the general formula (1):

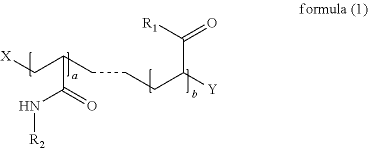

The two segments bearing the group R₁ and R₂, respectively, are randomly distributed over the length of the polymer chain.

In the above formula (1), X is either the initiator fragment derived by the radical starter molecule including but not exclusive 4′-azobis(4-cyanopentanoic acid) (ACPA), 2,2′-azobis(2-methylpropionitrile), 2-(azo (1-cyano-1-methylethyl))-2-methylpropane nitrile (AIBN) or can be a functional group (e.g., hydroxy, carboxylic acid, etc.) or a hydrogen atom.

Y can be a functional group (e.g., hydroxy, carboxylic acid, thiol, etc.), a hydrogen atom or the terminal RAFT agent used for further functionalization including but not exclusive 4-((((2-carboxyethyl)thio)carbonothioyl)thio)-4-cyanopentanoic acid (CTCTA) and can be chemically substituted or modified including but not exclusive by fluorophores carrying either a primary amine (e.g., fluoresceinamine) or a maleimide (e.g., 5-maleimido-fluorescein) or can be substituted by biomolecular tags like biotin or Rho1D4 Tag (e.g., biotin-maleimide, biotin-polyoxyethylen-maleimide, biotin-propargylamide).

The term functional group refers to any functional group known in the field of organic chemistry.

The letters a as well as b denote for the relative number of statistical distributed units of the polymer and give rise to the molecular weight of the polymer which can be in the range of 2.000 to 24.000 Dalton. a and b can be in the range from 0%-90% to the respective units and in general a+b=100% of the total functionalized and non-functionalized carboxylic acid groups of the polymer.

R₁ is the hydrophilic part of the polymer based on grafted proteinogenic and non-proteinogenic amino acids and amino acid derivatives and can be either a hydroxy group or an alkali metal (e.g., Li⁺, Na⁺, K⁺) ionically bound to an oxygen but can also be chemically modified and exchanged via amide formation with a phosphate- or sulfate-containing molecule including but not exclusive phosphorylethanolamine (2-aminoethyl dihydrogen phosphate) or homotaurine (3-aminopropane-1-sulfonic acid). Additionally, R₁ can be a polar amino acid with electrically charged side chains including but not exclusive aspartic acid, glutamic acid, as well as an amino acid with uncharged side chains including but not exclusive serine or threonine bound by amide formation. The grafting of the acrylic acid can be achieved by using organic or aqueous conjugation reactions including but not exclusive N-hydroxysuccinimide, 1-ethyl-3-(3-dimethylaminopropyl) carbodiimide, N,N′-dicyclohexylcarbodiimide, (benzotriazol-1-yloxytripyrrolidinophosphonium hexafluorophosphate), (2-(1H-benzotriazol-1-yl)-1,1,3,3-tetramethyluronium hexafluorophosphate as well as similar compounds and derivatives of said reagents.

R₂ is the hydrophobic part of the polymer and is derived from an amino acid. Examples for the described non-polar molecules can be phenylalanine benzyl ester (e.g., Sigma Aldrich, CAS 2462-32-0) or tryptophan benzyl ester (e.g., Sigma Aldrich, CAS 141595-98-4) whose carboxylic acid group can be chemically protected including but not exclusive by a methyl-, ethyl-, tert-butyl-, benzyl- or benzhydryl-ester. In addition to that, hydrophobic amino acids without protecting groups, such as phenylalanine, tryptophan, and tyrosine can be used.

### Definitions

1. The term “amphipol” refers to vinyl polymers which are capable of solubilizing membrane proteins and keep them stable in solution in their native form.
2. The term “nanodisc” refers to a small nanometer-sized complex formed by an amphiphilic polymer and lipids and is capable of stabilizing membrane proteins.
3. The term “vinyl” comprises, with respect to the present invention, the polymer backbone based on polymerization of ethenyl groups.
4. The term “membrane protein” comprises proteins which contain a hydrophobic transmembrane domain or are associated with the membrane by at least one hydrophobic domain. The membrane proteins can be monomeric or oligomeric and can be associated with cofactors.
5. The term “grafting” describes in the context of the present invention the functionalization of functional groups within the polymer chain with other molecules.
6. The term “dispersity” is expressed as polydispersity index (PDI) and can be calculated as the ratio of weight average by number to average molecular weight determined by aqueous size exclusion chromatography (SEC).
7. The term “Aminopol” refers to a polyacrylic acid backbone grafted with proteinogenic and non-proteinogenic amino acids with varying percentages.
8. The term “Phenylopol” refers to a polyacrylic acid backbone grafted with varying percentages of L-phenylalanine benzyl ester molecules.
9. The prefix “Phospho-” and “Sulfo-” refers to an polymer grafted with varying percentages of phosphate or sulfate containing molecules to enhance the solubility of the said polymer and enhance the resulting solubilization and stabilization efficiency during membrane protein solubilization and handling.
10. The term “affinity chromatography” is a method of separating a biomolecule from a mixture, based on a highly specific macromolecular binding interaction between the biomolecule and another substance. Affinity chromatography is useful for its high selectivity and resolution of separation, compared to other chromatographic methods. Examples for affinity chromatography are the purification of his- or rho-tagged proteins. These proteins have a poly-his (for example his6 or his10) or a TETSQVAPA amino acid sequence on the C or N terminus, allowing the purification from a mixture via selective binding onto Ni-NTA agarose (for his-tagged protein) or anti Rho-1D4 antibody agarose for rho-tagged proteins. Protocols for these purification procedures can be found on the Cube Biotech web site and in the literature of Hochuli et al. and Corin et al.

The present invention employs the use of polyacrylic acid (PAA) for the synthesis of an amphiphilic polymer. The PAA contain an alkyl backbone and side chains of carboxylic acid.

The PAA used for the present invention can be either purchased commercially (e.g., Thermo Scientific, Sigma Aldrich, CAS 79-10-7) or can be synthesized using radical polymerization, e.g., ATRP polymerization or RAFT polymerization [12], yielding a polymer with low polydispersity (PDI<2), more preferably with a pdi<1.2, even more preferably with a pdi<1.1, and terminal functionalizability. The described PAA is not limited to the described vendors, synthesis procedures or PDIs.

In case of ATRP polymerized polyacrylic acid, the synthesis is carried out using methyl acrylate monomers, an initiator and copper bromide as well as a catalytic agent. After polymerization, the resulting poly methyl acrylate polymer is hydrolyzed yielding the desired PAA. The ATRP-PAA is not limited to the described synthesis procedures.

In case of RAFT polymerized polyacrylic acid, the synthesis is carried out using a thiocarbonylthio compound, an initiator (e.g., thermochemical, photochemical initiator) and the acrylic acid monomer which is polymerized.

The molecular weight of the polymerized PAA can be in the range of 2-20 kDa but is not limited to the described range.

For generation of the amphiphilic character of the polymer according to the present invention, the polymer can be grafted using different techniques inclusively but not exclusively amide formation of the carboxylic groups of the PAA with a primary amine containing target molecule via carbodiimide-, isothiocyanate-, isocyanate-, acyl azide-, NHS ester-, anhydride, imidoester-, aldehyde- or epoxide-crosslinker chemistry.

The PAA grafting comprises the activation of the carboxylic groups and chemically modification using either crosslinking molecules or zero length crosslinker as well as chemically active target molecules (e.g., ester formation, acid halides formation).

The polymer class of PAA grafted with polar or non-polar as well as proteinogenic and non-proteinogenic amino acids and their derivatives are called “Aminopol” or are named by using an amino acid related prefix as in the case of PAA grafted with L-phenylalanine (Phenylopol).

The grafting of the PAA with polar molecules is in the range of >10%, preferred between 20-75% and mostly preferred between 25-50% but can be also in the range of 60-90%.

Polar molecules used for the grafting can be phosphate- or sulfate-containing molecules including but not exclusive phosphorylethanolamine (2-aminoethyl dihydrogen phosphate, e.g., Sigma Aldrich, CAS 1071-23-4) or homotaurine (3-aminopropane-1-sulfonic acid, e.g., Sigma Aldrich, CAS 3687-18-1). Additionally, polar proteinogenic amino acids and non-proteinogenic amino acids with electrically charged side chains including but not exclusive aspartic acid (e.g., Sigma Aldrich, CAS 56-84-8), Glutamic Acid (e.g., Sigma Aldrich, CAS 56-86-0), as well as an amino acid with uncharged side chains including but not exclusive Serine (Sigma Aldrich, CAS 56-45-1) or Threonine (e.g., Sigma Aldrich, CAS 80-68-2) as well as derivatives of the said molecules can be used.

The grafting of the PAA with non-polar molecules is in the range of >10%, preferred between 20-75% and mostly preferred between 25-50% but can be also in the range of 60-90%.

Non-polar molecules used for the grafting can be proteinogenic amino acids and non-proteinogenic amino acids whose carboxylic acid group were chemically protected including but not exclusive by using a methyl-, ethyl-, tert-butyl-, benzyl- or benzhydryl-ester as well as derivatives of the said molecules. Examples for the described non-polar molecules can be phenylalanine benzyl ester (e.g., Sigma Aldrich, CAS 2462-32-0) or tryptophan benzyl ester (e.g., Sigma Aldrich, CAS 141595-98-4). In addition to that, hydrophobic amino acids without protecting groups, such as phenylalanine, tryptophan, and tyrosine can be used. The grafting process can be achieved here with protected amino acids and successive removal of the protective group, or with a direct coupling with activated acrylic acid.

In the case of RAFT polymerized PAA, the terminal thiocarbonylthio moiety allows the functionalization of the polymer with target molecules inclusive but not limited to chromophores (e.g., fluorophores like fluorescein, or Alexa, or Raman active dyes like CY3 or CY5) or biomolecular tags (e.g., Biotin, Rho1D4, peptides). Substitution of the thiocarbonylthio moiety with the target molecule can be achieved inclusive but not exclusive using RAFT- or ATRP polymerization, thermolysis, radical-induced reduction, addition-fragmentation coupling, radical-induced oxidation or hetero Diels Alder reaction to give only few examples. A preferred way of modification is the reaction of the thiol function of the polymer with a maleimide-functionalized molecule, with a functionalized alkene, or with a functionalized alkyne.

In one embodiment, functional polymers can also be prepared by grafting of carboxy functions with functionalized amines, such as 5-aminofluorescein or amino biotin. The PAA grafting comprises the activation of the carboxylic groups and chemically modification using either crosslinking molecules or zero length crosslinker as well as chemically active target molecules (e.g., ester formation, acid halides formation).

There are at least two ways for the preparation:
Reaction of a mixture of one or more of the amino acids, like L-phenylalanin, with e.g. amino fluorescein to polyacrylic acid with e.g. EDC and HNS.
Reaction of e.g. amino fluorescein with a phenylopol, phospho-phenylopol or taurine phenylopol polymer by EDC/NHS activation.

Said terminal functionalized and terminal non-functionalized new amphiphilic polymers can be used for solubilization and stabilization of integral membrane proteins and membrane associated proteins.

The amphipols according to the present invention can be used for labelling of nanodisc complexes for their use in biomolecular research, diagnostic applications and medical product development.

To conclude the described invention, the new class of amphipols is based on:
1. Polyacrylic acid (PAA) synthesized via radical polymerization (e.g., atom transfer radical polymerization (ATRP), commercially available with variable molecular weight, e.g., Acros, Sigma Aldrich) or polyacrylic acid synthesized via reversible-addition-fragmentation chain-transfer (RAFT) polymerization. Noteworthy, the PAA synthesized with RAFT and used for the functionalization reveals a polydispersity index of <1.1.
2. Grafting of said PAA with polar and non-polar proteinogenic and non-proteinogenic amino acids and their derivatives as well as derivatives which bear chemically protected functional groups with and without their combination.
3. Optionally grafting of said new amphipols with or without molecules containing strongly charged or polar functional groups like sulfates and phosphates

Compared to the polymers of the prior art, the polymer according to the present invention has at least one of the following advantages:
1. Enhanced solubilization efficiency of membrane proteins expressed in procaryotic and eucaryotic cells and/or native proteins expressed in cell membranes.
2. Enhanced stability of formed nanodisc assemblies of membrane proteins during solubilization, purification and during application of said new amphipols and cell membrane patches with respect to high ionic strength and high concentrations of cations compared to commercially available products.
3. Due to the enhanced solubilization efficiency, the polymers according to the present invention can be used in lower concentrations, leading to higher purity and functionality of the stabilized membrane proteins.
4. Removement of excess polymer after solubilization by simple high-speed centrifugation for efficient binding of tagged membrane proteins or tagged copolymers to affinity resins and enabling advanced downstream applications.
5. Possibility of terminal functionalization of new amphipols synthesized with RAFT with molecules (e.g., fluorophores, biotin derivatives, etc.) using conjugation chemistry (e.g., maleimide, thiol-ene, thiol-yne click chemistry) for protein purification and protein labelling.
6. The high monodispersity of the polymers, a very narrow distribution of particle distribution with pdi values of smaller than 1.25, smaller than 1.2, or smaller than 1.1, which is obtained via RAFT or ATRP polymerization, results in more homogeneous complexes with membrane proteins and better results in analytics, such as x-ray structure analysis or cryo electron microscopy. These better results can be a higher resolution or a faster calculation time.

The solubilization, stabilization and purification of membrane proteins out of the native membrane surrounding is dependent on a number of parameters. Most parameters can be optimized during the purification process to a higher efficiency. The parameters include buffer conditions (for example salt, pH), choice of polymer, protein-to-solubilization agent-ratio, temperature, and time. First, cell lysis and centrifugation are carried out by for example using the following parameters: Adding of protease inhibitors (PI) to buffer and readjust pH value then disrupting cells (e.g., Sonification, French Press), centrifugation at 9 000 rcf for 30 min at 4° C., discarding pellet (cell debris, excess copolymer), collecting supernatant, centrifugation of the supernatant at 100 000 rcf for 1 h at 4° C., discarding supernatant and homogenize pellet. Then the solubilisation of membrane proteins is carried out: Polymers form synthetic nanodisc around the protein, thereby maintaining the native phospholipid environment and preserving the native and thus functional properties of the protein in a convenient one step manner (solubilization and stabilization). Detergents on the other hand form micelles around the hydrophobic belt, thus remove the lipids from the surrounding. For native conditions the unique lipid environment needs to be conserved.

In one embodiment, the membrane protein is selected from the group consisting of membrane receptor proteins, membrane enzymes, cell adhesion proteins, and transporter proteins, such as ABC transporters, ion channel proteins, water channel proteins (aquaporins), membrane-based ATPases, SLC transporters. That is, as a starting material for the method according to the present invention, a solution of the free polymer is used which stems from the solubilization, stabilization and purification of the above-mentioned membrane proteins out of their native surrounding by employing a polymer.

The amphiphilic polymers according to the present invention can be used for solubilization and stabilization of membrane proteins for biotechnological and pharmaceutical applications. Thus, it is also possible to use the amphiphilic polymer according to the present invention as reagent, in reagent kits and diagnostic kits (e.g., lateral flow assays) comprising at least one part of the described invention. One application of the amphipols according to the present is the solubilization and stabilization of membrane proteins in solution with and without detergent pretreatment. This means, that they are capable of keeping fully functional or non-functional but still immunogenic membrane proteins in solution and prevent them from aggregation or precipitation upon solubilization and handling. Thus, the invention is also related to the formed water-soluble complex consisting of one or more amphiphilic polymer molecules, artificial or natural lipids derived from cell membranes as well as integral membrane proteins or membrane associated proteins.

The use of polymers according to the present invention can be exemplified as follows:
1. One or more recombinant membrane or membrane associated proteins are expressed with high density in pro- or eucaryotic cells and are located either in or onto the cell membrane as well as potentially located in inclusion bodies.
2. The solubilizing step is carried out using either the whole cell suspension, the supernatant of cell lysate or the pellets of centrifuged supernatant derived from the cell lysate.
3. The protein solution or pellet is directly added to the polymer solution with a final polymer concentration up to 5% wt and incubated up to 24 h while stirring.
4. Solubilization efficiency can be determined using standard biomolecular methods (e.g., SDS-PAGE, Western Blot).
5. By Centrifugation, insolubilized proteins, debris as well as exess copolymer can be separated from the solubilized proteins located in the formed nanodisc complex.

In diagnostics, biological components such as DNA, RNA, proteins and metabolites are examined qualitatively and quantitatively. This provides information about diseases, genetic predispositions, or the state of health. Diagnostic tests can be performed by medical professionals, but also by private individuals.

The polymers according to the present invention can be used to solubilize and stabilize membrane proteins, preferably in their native lipid environment, in order to maintain their activity. These stabilized membrane proteins can be used to detect interactions. The interaction of the copolymer stabilized membrane protein and its interaction partner can be detected inclusive but not exclusively via different analytical methods.

Examples for optical detections contain SPR (surface plasmon resonance), RM (resonant mirror), GCI (Grating-Coupled Interferometry), ELISA (enzyme-linked immunosorbent assay) as Direct ELISA, Sandwich ELISA, Competitive ELISA, or Reverse ELISA, and LFA (lateral flow assay).

The interaction of the copolymer stabilized membrane protein and its interaction partner can be detected inclusive but not exclusively via different analytical methods.

Examples for optical detections contain SPR (surface plasmon resonance), RM (resonant mirror), GCI (Grating-Coupled Interferometry), ELISA (enzyme-linked immunosorbent assay) as Direct ELISA, Sandwich ELISA, Competitive ELISA, or Reverse ELISA, and LFA (lateral flow assay).

In addition to that, the copolymers can lyse eucaryotic cells and tissue in low concentration (0.01% to 5%) very quickly (in seconds to a few minutes) and without mechanical aids. Because of this capability for a mild lysis, it allows the user to obtain nucleic acids with a low level of fragmentation, and to get soluble and membrane proteins in their native state, the polymers of the invention are particularly suitable for diagnostic tests, especially basing on DNA, RNA, soluble proteins, and membrane proteins.

### BRIEF DESCRIPTION OF THE DRAWINGS

The invention is further illustrated by the following examples and figure. It is pointed out that the examples and figure are to be understood to illustrate the invention only and not to restrict the invention thereto.

FIG. 1 shows the size exclusion chromatograms of 0.5 wt % aqueous polyacrylic acid solution with different molecular weight and low PDI (<1.1) synthesized with RAFT polymerization.

FIG. 2 shows a UV-Vis extinction spectra of a 0.7 mg/ml Phenylopol solution in methanol

FIG. 3 shows the size exclusion chromatograms of 0.5 wt % Phenylopol fluorescently labelled with terminal conjugated fluorescein in DMF. The refractive index signal and fluorescence intensity signal is overlayed. The fluorescence intensity was multiplied with 50 For better visualization.

FIG. 4 shows the solubilization blot of a model solubilization of membrane proteins (G6PC) using different copolymers.

FIG. 5 shows photographs of: Phenylopol samples after solubilization of the membrane protein G6PC from cell lysate 1) before and 2) after high-speed centrifugation at 60.000 g for 1 h. Excess copolymer and cell debris is sedimented 3).

FIG. 6 shows UV-Vis spectra of the cell lysate with and without Phenylopol for membrane protein solubilization as well as before and after centrifugation to remove cell debris and excess copolymer.

FIG. 7 shows the isolation blot of a model purification of membrane proteins (G6PC) using different copolymers and Rho affinity resin purification.

### DETAILED DESCRIPTION

The present invention is described in the following by examples. It is explicitly pointed out that the examples shall not be construed to limit the invention thereto.

### 1. Synthesis of Monodisperse Polyacrylic Acid (PAA) Using RAFT Polymerization:

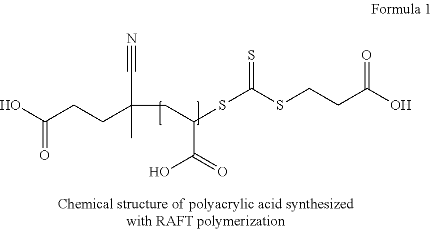

The synthesis of polyacrylic acid (PAA) via RAFT polymerization was performed following a modified protocol derived by Chaduc et al. (2013). The targeted molecular mass of the PAA polymer was varied between 2 kDa and 15 kDa via varying the amount acrylic acid used for the synthesis. The synthesis of 5 kDa PAA was performed by mixing of 1.87 g (26 mmol) acrylic acid with 20 mg 4,4′-Azobis(4-cyanopentanoic acid) (ACPA), 149 mg (0.49 mmol) CTCTA and 8 mL of deionized water in a 50 ml round-bottom flask with ground-glass stopper. For synthesis of 2, 3, 4, 7, 9, 11 and 15 kDa PAA, 0.75, 1.15, 1.5, 1.87, 2.8, 3, 4.3, 5.9 g acrylic acid were added, respectively. After dissolution of the solid educts the resulting yellow solution was deoxygenized by argon-bubbling for 30 min. Afterwards the flask was sealed and incubated at 70° C. for 24 h. The crude PAA product was purified by precipitation of the PAA in 10 mL diethyl ether prior to the functionalization with ligands. The molecular weight of the polymer was determined using aqueous size-exclusion chromatography (Agilent Infinity 1260, 2× aquagel-OH 20 columns, 200 mM NaNO₃, 20 mM Na₂HPO₄, pH 7.5).

Aim of the polyacrylic acid synthesis via RAFT polymerization is to obtain a polymer with a low polydispersity index as well the capability of precise terminal functionalization. The determination of the molecular weight of the polyacrylic acid synthesized with said RAFT polymerization was performed via size-exclusion chromatography. The results reveal highly monodisperse polymers with a PDI below 1.1.

Additionally, the size of the PAA can be precisely varied in a broad range molecular weight relevant for the solubilization of membrane proteins.

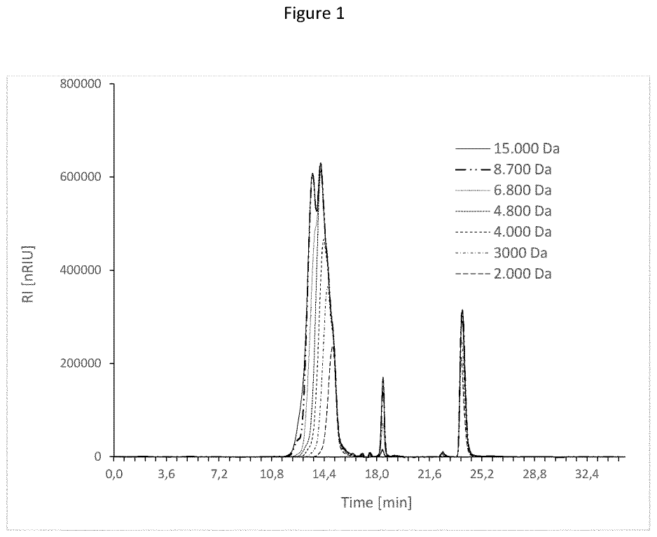

FIG. 1 shows the size exclusion chromatograms of polyacrylic acid with different molecular weight and low PDI (<1.1) synthesized with RAFT polymerization. Size-characterization was performed using an Agilent Infinity II 1260 system including an isocratic pump, a column oven and a refractive index detector. For separation two Agilent Aquagel-OH 20 columns were used as well as mobile phase containing sodium nitrate (200 mM) and sodium phosphate (20 mM, pH 7.5). As molecular weight standard, the polyacrylic acid calibration kit (Agilent) was used.

### 2. Synthesis of Polyacrylic Acid Using ATRP Polymerization[11]

54.1 mg (0.38 mmol) CuBr, 2 mL (22.2 mmol) methyl acrylate, 0.08 mL (0.38 mmol) N,N,N′,N″,N″-pentamethyldiethylenetriamine (PMDETA) and 0.0 mL (0.45 mmol) methyl 2-bromopropionate (2-MBP) were mixed in a 50 ml round-bottom flask with ground-glass stopper and deoxygenized by argon-bubbling for 30 min followed by incubation at 50° C. for 4.5 h. The crude PAA product was purified by precipitation of the PAA in 10 mL diethyl ether prior to the functionalization with ligands. The hydrolysis of the PMA backbone was achieved by dissolution of 0.2 g of PMA in 20 mL THF, adding of 25 mL sodium hydroxide solution (1.37 g) and incubation at 60° C. for 10 h. The molecular weight of the polymer was determined using aqueous size-exclusion chromatography (Agilent Infinity 1260, 2× aquagel-OH 20 columns, 200 mM NaNO₃, 20 mM Na₂HPO₄, pH 7.5).

3. Functionalization of PAA with L-Phenylalanine Benzyl Ester

Briefly, 1 g of the synthesized PAA (~6 kDa) mentioned above or alternatively commercially purchased was mixed with 2 g (6.6 mmol) L-Phenylalanine benzyl ester hydrochloride and 1.3 g (14.18 mmol) N-hydroxysuccinimide in 50 mL phosphate buffer (100 mM, pH 7.2) and 50 mL Methanol. While stirring, 0.5 g 1-Ethyl-3-(3-dimethylaminopropyl) carbodiimide (EDC) is added and the resulting mixture is stirred for 30 min. The last step is repeated three times and the reaction mixture is incubated for 24 h. Afterwards, the crude reactions mixture is purified using dialysis against distilled water using a 1 kDa dialysis tube until the conductivity of the water has reached <10 μS. The solid polymer called hereinafter in the following “Phenylopol” was isolated by using vacuum drying. The grafting percentage was determined using titration with hydrochloric acid giving the amount of remaining free carboxylic acid groups. The functionalization of the polymer was determined using size-exclusion chromatography (Agilent Infinity 1260, 1× PlGel mixed column, DMF, 0.05% LiBr).

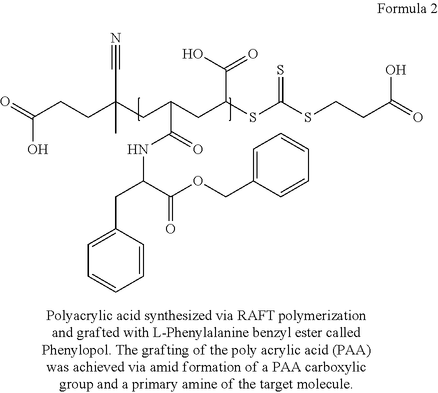

### 4. Deprotection of Phenylopol Using Base Catalyzed Hydrolysis of the Benzyl Ester

The deprotection was carried out using 1 g (0.17 mmol) of Phenylopol grafted with 50% and a five-fold molar excess of (1.4 g, 35 mmol) NaOH dissolved in 30 mL deionized water with respect to the benzyl ester moieties. The resulting solution was heated for 10 min to 100° C. under reflux and purified using dialysis.

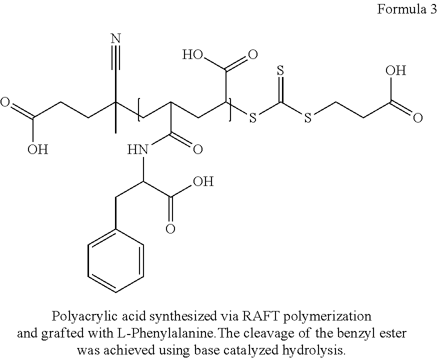

5. Synthesis of Phospho-Phenylopol by Functionalization of Phenylopol with Phosphorylethanolamine

The functionalization of Phenylopol with strongly charged functional groups, e.g., phosphates or sulphates was carried out as described using the example of phosphorylethanolamine. Briefly, 1 g of Phenylopol was mixed with 3.25 g (20 mmol) phosphorylethanolamine and 1.1 g (17.7 mmol) N-hydroxysuccinimide in 50 ml (100 mM, pH 7.2) phosphate buffer. While stirring, 0.5 g 1-Ethyl-3-(3-dimethylaminopropyl) carbodiimide (EDC) is added and the resulting mixture is stirred for 30 min. The last step is repeated three times and the reaction mixture is incubated for 24 h. Afterwards, the crude reactions mixture is purified using dialysis against distilled water using a 1 kDa dialysis tube until the conductivity of the water has reached <10 μS. The solid polymer called Phospho-Phenylopol was isolated by using vacuum drying. The grafting percentage was determined using titration with hydrochloric acid giving the amount of remaining free carboxylic acid groups (<10%).

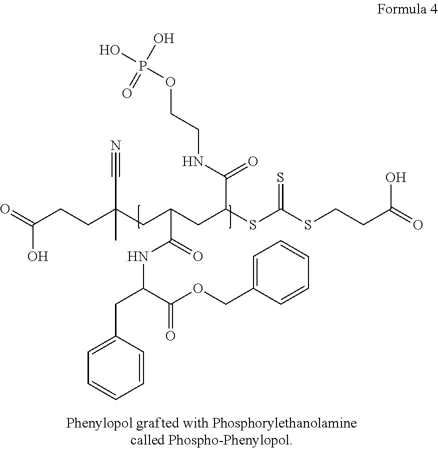

The terminal functionalization of RAFT polymers was performed via three different mechanisms:
a) The generation of reduced terminal thiol group via decomposition of the RAFT agent thiocarbonate using butylamine and the covalent coupling of the reduced thiol with a biological tag or a fluorophore containing a maleimide moiety.
b) The conjugation of an amine containing fluorophore or biological tag with a heterobifunctional linker molecule (e.g., 1-[(4-([(2,5-Dioxo-1-pyrrolidinyl)oxy]-carbonyl)cyclohexyl)methyl]-1H-pyrrol-2,5-dion (SMCC)) followed by conjugation to the reduced terminal thiol of the decomposed thiocarbonate as described in 1.
c) The third mechanism is the application of thiol-yne or thiol-ene Click-chemistry.[13]

### 6. Conjugation of Phenylopol, Synthesized Via RAFT Polymerization, to Fluorescein-Maleimide

Briefly, 1 g Phospho-Phenylopol, 74 mg Tris(2-carboxyethyl)phosphine (TCEP) and 125 mg Fluorescein-Maleimide were dissolved in 20 mL phosphate buffer (100 mM, pH 7.0) under stirring followed by the addition of 210 mg butylamine. The resulting reaction mixture was incubated for 48 h. Afterwards, the dissolved polymer was purified and isolated using dialysis and vacuum drying as described before. The functionalization of the polymer was determined using size-exclusion chromatography (Agilent Infinity 1260, 1× PlGel mixed column, DMF, 0.05% LiBr)

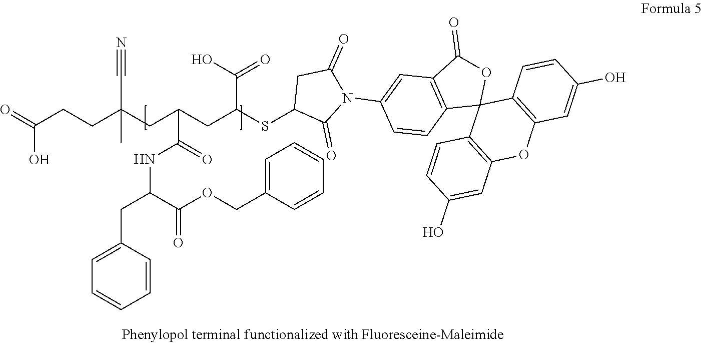

7. Conjugation of Phenylopol, Synthesized Via RAFT Polymerization, with Amino-Fluorescein Using a Heterobifunctional Crosslinker

Briefly, 396 mg (1.14 mmol) Amino fluorescein and 191 mg (0.57 mmol) SMCC were dissolved in 40 mL Dimethylformamide and 10 ml Methanol and incubated for three hours at room temperature. Afterwards, 1 g Phospho-Phenylopol was added and dissolved under stirring followed by the addition of 166 mg (2.29 mmol) butylamine and incubation for 24 hours on an end-over-end shaker. The dissolved polymer was purified and isolated using dialysis and vacuum drying as described before.

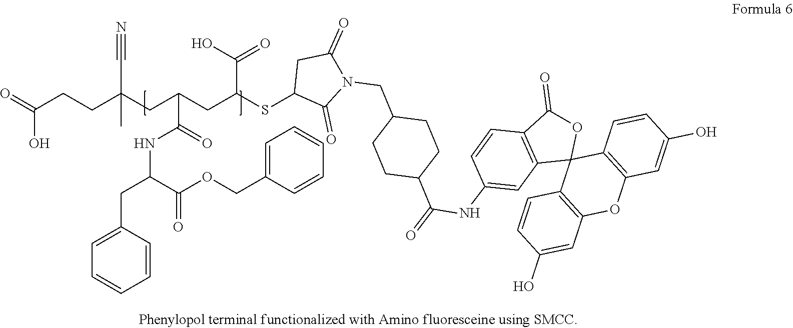

### 8. Conjugation of Phospho-Phenylopol, Synthesized Via RAFT Polymerization, to Biotin-Propargylamide Via Thiol-Ene Click Chemistry [10, 14]

Briefly, 1 g (77 μmol) Phospho-Phenylopol, 2.2 mg 4,4′-Azobis(4-cyanopentanoic acid) (ACPA) (7.7 μmol) and 109 mg (388 μmol) Biotin-Propargylamide were dissolved in 10 ml deionized water in a 50 ml round-bottom flask with ground-glass stopper. The reaction mixture was deoxygenized by bubbling with Argon for 30 min, heated to 70° C. and incubated for 24 h. Afterwards, the crude product was purified using dialysis as described before and vacuo dried.

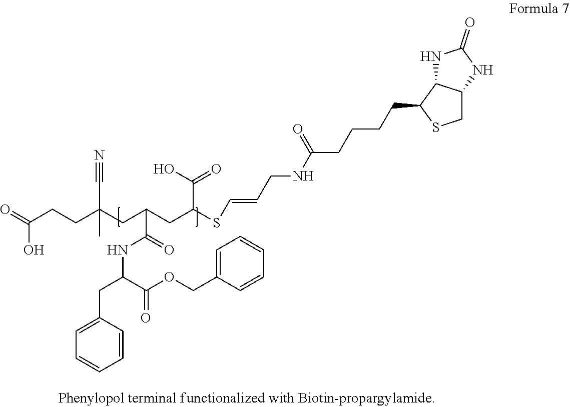

9. Solubilization of his- or Rho-Tagged Membrane Protein G6PC and Purification

The solubilization, stabilization and purification of membrane proteins out of the native membrane surrounding is dependent on a number of parameters. Most parameters can be optimized during the purification process to a higher efficiency. Parameters include: Buffer conditions (salt, pH etc.), choice polymer, protein-to-solubilization agent-ratio, temperature, time.

### Cell Lysis and Centrifugation:

Add protease inhibitors (PI) to buffer and readjust pH value then disrupt cells (e.g., Sonification, French Press). Centrifuge at 9 000 rcf for 30 min at 4° C., discard pellet (cell debris), collect supernatant. Centrifuge supernatant at 100 000 rcf for 1 h at 4° C., discard supernatant and homogenize pellet.

### Solubilization of Membrane Proteins:

Polymers form synthetic nanodisc around the protein, thereby maintaining the native phospholipid environment and preserving the native and thus functional properties of the protein in a convenient one step manner (solubilization and stabilization). Detergents on the other hand form micelles around the hydrophobic belt, thus remove the lipids from the surrounding. For native condition the unique lipid environment needs to be conserved.

If solubilization efficiency is low it is advised to screen variation of parameters to improve the yield of total solubilized protein. A standard protocol is described as follows:
Add solubilization agent (copolymer) to the protein solution
Ideal concentrations may vary, good starting points are:
0.1-2.5% copolymer (e.g. Aminopol, Phenylopol-Fluorescein)
Solubilize for 3 h to 24 h at 4° C. while stirring
Higher temperatures can be screened for optimization
Centrifuge at 100 000 rcf for 1 h at 4° C.
Discard the pellet containing cell fragments and excess polymer, collect the supernatant
Use solubilized membrane protein in polymer nanodisc (supernatant) for affinity chromatography, separating the his- or rho-tagged membrane protein copolymer complex from the mixture by using commercially available agarose products.
Visualization of solubilized membrane proteins by SDS-PAGE, Western Blot and Rho-Antibody/HRP staining for chemiluminescence detection

A further standard protocol is described as follows:
Add solubilization agent (copolymer) to the protein solution
Ideal concentrations may vary, good starting points are:
0.1-2.5% Phenylopol
Solubilize for 3 h to 24 h at 4° C. while stirring
Higher temperatures can be screened for optimization
Centrifuge at 100 000 rcf for 1 h at 4° C.
Discard the pellet, collect the supernatant
Use solubilized membrane protein in polymer nanodisc (supernatant) for affinity chromatography, separating the his- or rho-tagged membrane protein copolymer complex from the mixture.

### Results:

The first step in producing a well-defined copolymer grafted with amino acids is the synthesis of a well-defined acrylic acid polymer with different molecular weights as described before. FIG. 1 shows the size exclusion chromatograms of polyacrylic acid polymers with varied molecular length. To ensure a low polydispersity, in this example RAFT polymerization was used. The molecular weight of the resulting polyacrylic acid can be exemplary varied between 2 kDa and 15 kDa with a polydispersity below 1.2 by changing the amount of acrylic acid added to the polymerization solution. With increasing amount of monomers present in the polymerization solution, the molecular weight of the final polymer is increasing as evident from FIG. 1. With decreasing molecular weight, the retention time of the polymer during the size exclusion chromatography is prolonged as the polymer interacts more with the size exclusion column. For the determination of the molecular weight, a polyacrylic acid calibration kit (Agilent) was used.

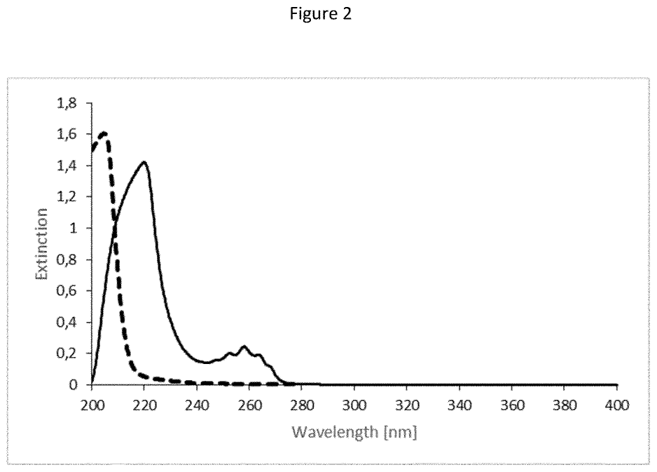

The synthesis of Phenylopol was exemplary achieved by 40 mol % grafting of polyacrylic acid using L-Phenylalanine benzyl ester. After purification and drying, the resulting copolymer reveals strong absorption modes with peak absorption at 258 nm as evident in FIG. 2 (solid line). As control, polyacrylic acid was measured (dashed line) which reveal no distinct absorption modes at 260 nm. The increasing absorption at 220 nm of Phenylopol is contributed by the polyacrylic acid-amide backbone of the polymer.

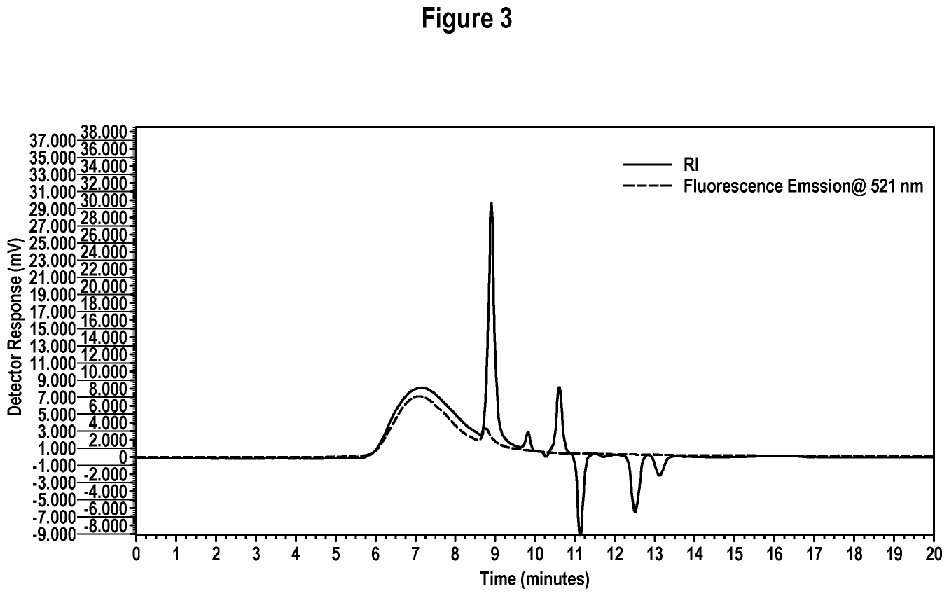

The further functionalization of the previously synthesized Phenylopol is exemplary showed by using a terminal modification with amino fluorescein and SMCC. For analyzation of the resulting fluorescently labelled polymer, organic phase size exclusion chromatography was used. FIG. 3 shows the respective chromatogram with an overlay of the refractive index detection as well as the corelated fluorescence detection (excitation 489 nm, emission 521 nm). Due to the more hydrophobic character of the polymer after the functionalization with fluorescein and L-Phenylalanine benzyl ester, the polymer was analysed using DMF as mobile phase with 0.05 wt % LiBr. For better visualization, the fluorescence signal was multiplied by fifty. The Phenylopol polymer peak located at ~7 min reveals a fluorescence signal which confirmed the successful functionalization. The second peak located at ~9 min corresponds to traces of unconjugated SMCC-fluoresceine.

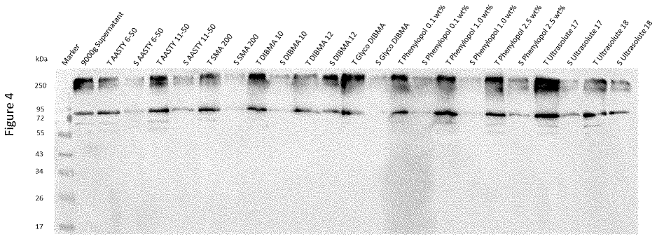

Exemplary, Phenylopol grafted with 40% was used to solubilize the model membrane protein G6PC derived by HEK cells. G6PC was overexpressed in the cell line by transformation. Sodium dodecylsuphate polyacrylaminde gel electrophoresis (SDS-PAGE) was used for the analysis and size separation of the cellular expressed proteins with respect to their molecular weight. FIG. 4 shows the results of the membrane solubilization on SDS-PAGE using a western blot, primary labelling using a primary anti-Rho tag antibody and a secondary HRP conjugated antibody for chemiluminescence detection. Successful solubilization of G6PC produces a band with a molecular weight around 80 kDa as evident through the broad range marker. To compare the solubilization efficiency, the crude cell lysate as well as different state-of-the-art copolymers commercially available are used as controls. For every polymer the crude solubilizate (T) as well as the centrifuged (100.000 rcf, 1 h) supernatant is analyzed in the gel. The performance of Phenylopol can be compared to the most efficient commercially available copolymers (e.g. Ultrasolute 17 and 18) where dark and distinct bands at ~80 kDa indicates a high purity and a high yield solubilization.

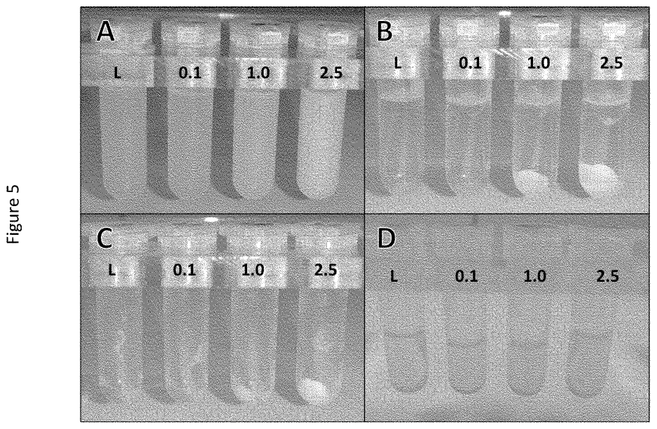

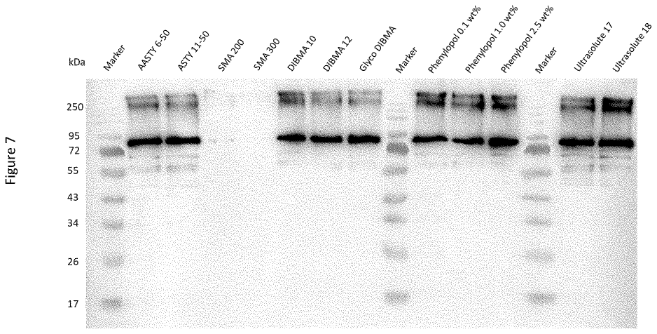

The stable and efficiently isolation of the solubilized target protein using affinity resin purification is maybe the most important step in membrane protein. Removing excess copolymer present in the solubilizate before affinity purification can dramatically enhance the quality of the isolated protein as the binding of the affinity tag to the affinity resin (e.g. His-Tag to NTA resins) can be strongly influenced and hindered by excess copolymers. Thus, removal of excess polymer is highly needed. Phenylopol bears the unique property, as the excess polymer can be removed by simple centrifugation before binding of the tagged protein to an affinity resin as shown in FIG. 5. FIG. 5 shows centrifugation tubes containing cell lysate and cell lysate (L) solubilized with 0.1, 1.0 and 2.5 wt % Phenylopol, respectively. The turbidity of the cell lysate is reduced by addition of 0.1 wt % of Phenylopol which is an indication of membrane solubilization. With increasing polymer wt % the turbidity increases as excess Phenylopol not forming nanodiscs is suspended in the cell lysate (A). By using centrifugation, the excess polymer as well as not solubilized cell membranes can be sedimented and removed from the supernatant prior to affinity resin binding (B, C, D). FIG. 5, B shows the sedimented not solubilized cell membrane (yellowish) as well as the excess polymer (white) after centrifugation. The importance of this feature is illustrated by comparing the blot intensities of the S Phenylopol 0.1 wt % (FIG. 4) and the S Ultrasolute 18 sample. The G6PC blot intensity is stronger in the S Ultrasolute 18 sample. However, the isolation of G6PC using Rho-affinity purification reveals comparable blot intensities with less carry-over of off-target proteins while initially using 25fold less copolymer (FIG. 7).

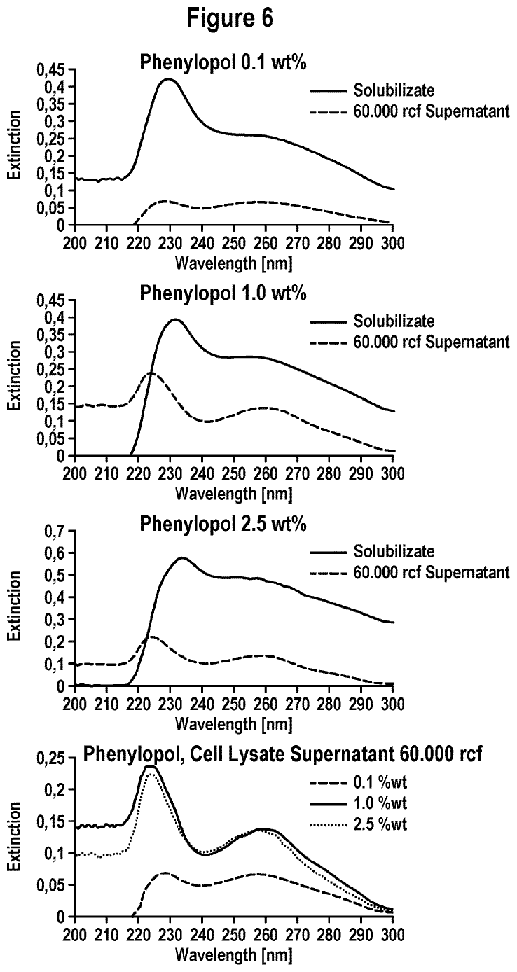

The analyzation of the cell lysate as well as cell lysate/Phenylopol samples using UV-Vis extinction spectroscopy reveal an efficient removal of not solubilized cell membranes, proteins and excess copolymer (FIG. 6). To compensate the contribution of the cell membrane and not soluble proteins, every sample was blanked with the respective cell lysate and the cell lysate 60.000 rcf control. A decreasing absorption of the L-Phenylalanine benzyl ester at 260 nm wavelength indicates the removal of excess polymer. Furthermore, the analysis of the supernatant enables the identification of the polymer concentration for the most efficient solubilization of cell membranes (FIG. 6, Phenylopol, Cell Lysate Supernatant 60.000 rcf).

Both, the more efficient solubilization with respect to the isolation efficiency as well as the removal of excess copolymer prior to affinity resin purification is a key feature of the new invented polymer series exemplary shown by using Phenylopol for membrane protein isolation. Advantageously, the physicochemical properties of the Aminopol polymer series can be varied by using different protected and non-protected amino acids for polyacrylic acid grafting which making the Aminopol series suitable for different membrane proteins as well as downstream applications.

Furthermore, the use of polymerization techniques like RAFT polymerization allows the synthesis of a well-defined polymer with an extremely narrow polydispersity as well as the chain-end functionalization described before and exemplary shown in the results. This feature is not be given by current commercially available polymers (e.g. DIBMA, SMA, Ultrasolute).

The foregoing description is for the purpose of exemplary illustration and is not intended to be exhaustive or to limit the invention the precise forms disclose.

Summing up, the present invention relates to polymers of general formula (1) based on acrylic acid which is functionalized with polar and non-polar, chemically protected proteogenic and non-proteogenic amino acids as well charged molecules like phosphorylethanolamine or homotaurine for enhanced solubilization efficiency, stability of the formed complexes and resistance against high ionic strength. Amphiphilic polymers according to the present invention are used for solubilization and stabilization of membrane associated proteins in aqueous media and the use of the membrane protein-polymer complex in biotechnological and medical applications. Furthermore, the new developed amphiphilic polymers bear the feature of further chemically labeling with molecular tags and dyes for protein labelling.

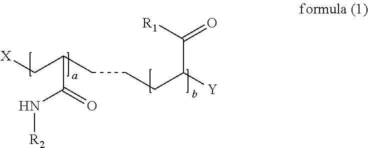

### REFERENCES

1. Rabilloud, T., Membrane proteins and proteomics: love is possible, but so difficult. Electrophoresis, 2009. 30 Suppl 1: p. S174-80.
2. Marconnet, A., et al., Solubilization and stabilization of membrane proteins by cycloalkane-modified amphiphilic polymers. Biomacromolecules, 2020: p. 3459-3467.
3. Smith, A. A. A., et al., Lipid Nanodiscs via Ordered Copolymers. Chem, 2020. 6(10): p. 2782-2795.
4. Zoonens, M. and J. L. Popot, Amphipols for each season. J Membr Biol, 2014. 247(9-10): p. 759-96.
5. Timcenko, M., A. A. A. Autzen, and H. E. Autzen, Characterization of Divalent Cation Interactions with AASTY Nanodiscs. ACS Applied Polymer Materials, 2022. 4(2): p. 1071-1083.
6. Dahmane, T., et al., Sulfonated amphipols: synthesis, properties, and applications. Biopolymers, 2011. 95(12): p. 811-23.
7. Zampieri, V., et al., CryoEM reconstructions of membrane proteins solved in several amphipathic solvents, nanodisc, amphipol and detergents, yield amphipathic belts of similar sizes corresponding to a common ordered solvent layer. Biochim Biophys Acta Biomembr, 2021. 1863(11): p. 183693.
8. Smith, A. A. A., et al., Controlling Styrene Maleic Acid Lipid Particles through RAFT. Biomacromolecules, 2017. 18(11): p. 3706-3713.
9. Chiefari, J., et al., Living Free-Radical Polymerization by Reversible Addition-Fragmentation Chain Transfer: The RAFT Process. Macromolecules, 1998. 31: p. 5559-5562.
10. Moad, G., E. Rizzardo, and S. H. Thang, End-functional polymers, thiocarbonylthio group removal/transformation and reversible addition-fragmentation-chain transfer (RAFT) polymerization. Polymer International, 2011. 60(1): p. 9-25.
11. Peng, D., et al., Synthesis of poly(acrylic acid)-g-polystyrene copolymer by successive ATRP. Polymer Bulletin, 2010. 65(7): p. 657-667.
12. Chaduc, I., et al., Effect of the pH on the RAFT Polymerization of Acrylic Acid in Water. Application to the Synthesis of Poly (acrylic acid)-Stabilized Polystyrene Particles by RAFT Emulsion Polymerization. Macromolecules, 2013. 46(15): p. 6013-6023.
13. Fairbanks, B. D., D. M. Love, and C. N. Bowman, Efficient Polymer-Polymer Conjugation via Thiol-ene Click Reaction. Macromolecular Chemistry and Physics, 2017. 218(18).
14. Oishi, E., M. Takamura, and T. Takahashi, Removal of Trithiocarbonyl End Group of RAFT-Polymerized Poly(stearyl acrylate) and Effect of the End Group on Thermal and Structural Properties. Polymers (Basel), 2021. 13(23).
15. His affinity purification: Hochuli, E., Bannwarth, W., Döbeli, H. et al. Genetic Approach to Facilitate Purification of Recombinant Proteins with a Novel Metal Chelate Adsorbent. Nat Biotechnol 6, 1321-1325 (1988)
16. Rho affinity purification: Corin, K., Baaske, P., Geissler, S. et al. Structure and function analyses of the purified GPCR human vomeronasal type 1 receptor 1. Sci Rep 1, 172 (2011). https://doi.org/10.1038/srep00172

1. A water-soluble membrane protein solubilizing polymer having the general formula (1) 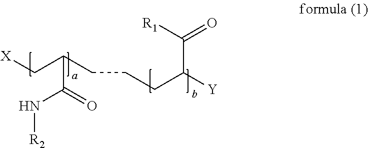
wherein
X is either the initiator fragment derived by the radical starter molecule used for preparing the polymer or a functional group or a hydrogen atom;
Y is a functional group, a hydrogen atom or a terminal RAFT agent usable for further functionalization and can also be conjugated with other molecules like fluorophore, and biomolecular tag;
a as well as b denotes for the relative number of statistically distributed units of the polymer and give rise to the molecular weight of the polymer;
R₁ is a hydrophilic part of the polymer based on grafted proteinogenic and non-proteinogenic amino acids and amino acid derivatives, and polar functional groups like Phosphoethanolamin and derivates;
R₂ is a hydrophobic part of the polymer based on grafted proteinogenic and non-proteinogenic amino acids and amino acid derivatives.

2. The polymer according to claim 1, wherein the initiator fragment is selected from the group consisting of 4′-azobis(4-cyanopentanoic acid), 2,2′-azobis(2-methylpropionitrile), and 2-(azo (1-cyano-1-methylethyl))-2-methylpropane nitrile.

3. The polymer according to claim 1, wherein the RAFT agent is selected from the group consisting of 4-((((2-carboxyethyl)thio)carbonothioyl)thio)-4-cyanopentanoic acid and can be chemically substituted or modified including but not exclusive by fluorophores carrying either a primary amine or a maleimide or can be substituted by biomolecular tags.

4. The polymer according to claim 1, wherein the molecular weight is from 2.000 to 24.000 Dalton.

5. The polymer according to claim 1, wherein a and b are in the range from 0%-90% to the respective units and in general a+b=100% of the total functionalized and non-functionalized carboxylic acid groups of the polymer.

6. The polymer according to claim 1, wherein the hydrophilic part is selected from the group consisting of a hydroxy group, an alkali metal ionically bound to an oxygen, which can also be chemically modified and exchanged via amide formation with a phosphate- or sulfate-containing molecule, a mono or poly amino acid with electrically charged side chains or an amino acid with uncharged side chains or a combination.

7. The polymer according to claim 1, wherein the hydrophobic part is a proteinogenic or non-proteinogenic amino acid whose carboxylic acid group was chemically protected.

8. The polymer according to claim 7, wherein the protected amino acid is selected from the group consisting of phenylalanine and tryptophan, protected with groups like benzyl ester and tert butyl ester.

9. The polymer according to claim 1, wherein the hydrophobic part is an amino acid.

10. The polymer according to claim 9, wherein the amino acid is phenylalanine or tryptophan.

11. The polymer according to claim 1, wherein the polydispersity index of the polymer, defined as the ratio of weight average by number to average molecular weight determined by aqueous size exclusion chromatography (SEC), is 1.25 or less.

12. The polymer according to claim 11, wherein the polydispersity index of the polymer is 1.1 or less.

13. The polymer according to claim 1, wherein the functional group introduced to position Y is biotin, a peptide, a tag, a fluorophore, or a dye.

14. A method for preparing a polymer as defined in claim 1, wherein a polyacrylic acid polymer is prepared and functionalized to introduce the substituents R₁, and/or R₂.

15. Use of a polymer for solubilization and stabilization of membrane proteins, comprising treating the membrane proteins with the polymer of claim 1.

16. A water-soluble membrane protein amphiphilic vinyl polymer complex comprising the polymer of formula 1.

17. The complex of claim 16 further comprising lipid compounds.

18. A method of preparing the water-soluble membrane protein-amphiphilic vinyl polymer complex according to claim 16 comprising a solution step in which a protein fraction from a biological or synthetic membrane containing said membrane protein, or a mixture of membrane proteins is brought into contact with the polymer of formula 1.

19. (canceled)

20. A kit for the solubilization and stabilization of membrane proteins containing the polymer as defined in claim 1.

21. A kit for eucaryotic cell or tissue lysis, containing one or more polymers of claim 1.
